(19) United States
(12) Patent Application Publication
(10) Pub. No.: US 2016/0206228 A1
(43) Pub. Date: Jul. 21, 2016

(54) Endoscope Tracking Device

(71) Applicants: Leticia M. ANGULO, Calgary (CA); Christopher N. ANDREWS, Calgary (CA); Christopher A. SIMON, Calgary (CA); Martin P. MINTCHEV, Calgary (CA); UTI LIMITED PARTNERSHIP, Calgary (CA)

(72) Inventors: Leticia M. Angulo, Calgary (CA); Christopher N. Andrews, Calgary (CA); Christopher A. Simon, Calgary (CA); Martin P. Mintchev, Calgary (CA)

(73) Assignee: UTI Limited Partnership, Calgary, AB (CA)

(21) Appl. No.: 14/908,590

(22) PCT Filed: Jul. 28, 2014
(86) PCT No.: PCT/IB2014/002364

§ 371 Date: Jan. 29, 2016

Related U.S. Application Data
(60) Provisional application No. 61/859,741, filed on Jul. 29, 2013.

(51) Int. Cl. A61B 5/06; A61B 1/04; A61B 1/00

(52) U.S. Cl. CPC A61B 5/065; A61B 1/00009; A61B 1/04; A61B 1/00045; A61B 1/00055; A61B 2090/066

(57) ABSTRACT

The present invention provides an external apparatus that allows automated quantification of the quality of the endoscopy procedure and methods for using the same. The apparatus of the invention includes an endoscope tracking device that is operatively connected to an endoscope cord surface so that a variety of information related to movement of the endoscope can be accurately captured. Exemplary movement information include, but are not limited to, distance travelled by the endoscope, direction of movement by the endoscope, torque exerted by the endoscope, etc. The endoscope tracking device comprises an electromechanical tracking sensor(s) that produce movement information of the endoscope including the ratio of forward and backward movements as well as withdrawal time of the endoscope. As these metrics can be obtained automatically, the apparatus and the method of the present invention can be used as a quality control in daily medical practice. Furthermore, the apparatus and the method of the invention can be used to create or determine best practice standards for endoscopy procedure training or as part of medical skills evaluation.

### CROSS-REFERENCE TO RELATED APPLICATIONS

This application claims the priority benefit of U.S. Provisional Application No. 61/859,741, filed Jul. 29, 2013, which is incorporated herein by reference in its entirety.

### FIELD OF THE INVENTION

The present invention relates to an electromechanical system for real time measuring of quality parameters in a colonoscopy video.

### BACKGROUND OF THE INVENTION

Colorectal cancer (CRC) is one of the leading causes of cancer mortality. Colonoscopy is the most accurate method of screening the colon for colorectal cancer. However, its effectiveness is dependent on the quality of the procedure. Due to variations in clinical practice of individuals that conduct colonoscopy, the accuracy and result can vary significantly. Image quality of colonoscopy depends inter alia on withdrawal time and velocity. These factors determine the image quality of colonoscopy, and hence the usefulness of the procedure. In fact, inability to consistently intubate the caecum, and rapid withdrawal times are significant contributors to missed lesions during colonoscopy.

Because the colonoscopy quality is a major factor in detecting polyps in CRC screening, there is a tremendous interest in the creation and evaluation of quality metrics for colonoscopy.

### SUMMARY OF THE INVENTION

Some aspects of the invention provide a device that tracks an endoscope (e.g., a colonoscope) movement to provide quality metrics for endoscopy. An endoscope is a device that is used to look inside a body cavity or organ. Typically, the endoscope is inserted through a natural opening, such as the mouth during a bronchoscopy or the rectum for a sigmoidoscopy or colonoscopy.

In some embodiments, the device is a one-piece miniature device that tracks the endoscope movement. Some of the advantages of the device of the invention include, but are not limited to, (i) the device is placed externally to the endoscope, and therefore no modification of existing endoscope is needed; (ii) the device is adaptable for various endoscopes such as colonoscope, bronchoscope, gastroscope, laryngoscope, cystourethroscope, esophagogastroduodenoscope, proctosigmoidoscope, etc.; and (iii) withdrawal time can be readily calculated after detecting a boundary between the insertion and withdrawal phases of the endoscope as well as other advantages as described herein.

In other embodiments, the device includes an electromechanical tracking system. Such tracking system can be configured to simply count the positive (e.g., for forward motion) and negative (e.g., for backward motion) pulses generated by the motion of the endoscope. The device is also configured to track the distance or the length of endoscope movement as well as the time. In this manner, the velocity or speed of the endoscope movement can be calculated by a simple division of the endoscope travel distance by the time of travel. The calculated velocity or the speed assessment can be used as a real-time feedback for the clinician or the doctor as a quality metric for endoscopy test. Moreover, the velocity or the speed of endoscope movement can be used as a quality metric for the video frames. For example, a relatively fast endoscope insertion or withdrawal often leads to blurry images; thus, one can evaluate the image quality based on the velocity of endoscope movement.

Yet in other embodiments, the device is also configured to provide various statistics such as withdrawal time, % of time with adequate visualization (e.g., % of time with the endoscope velocity within an acceptable range), forward and backward movement statistics, etc. Such a summary of statistics can be used by the clinician or the doctor to evaluate the reliability of endoscope result or it can be used to alert the clinician or the doctor of a possibility of unreliability of endoscopy images.

One particular aspect of the invention provides an electromechanical tracking device configured for use in an endoscopy procedure, said electromechanical tracking device comprising: (i) a rotatable wheel configured such that an endoscope is adapted to be in contact with said rotatable wheel, wherein said rotatable wheel is further configured to rotate in proportion to the amount of movement of the endoscope that is in contact with said rotatable wheel; (ii) an electromechanical sensor operatively connected to said rotatable wheel, wherein said electromechanical sensor is configured to generate a first signal when said rotatable wheel moves in one direction and a different signal when said rotatable wheel moves in an opposite direction; and (iii) an electrical signal receiving unit that is configured to receive the electric signal generated by said electromechanical sensor, wherein said electrical signal receiving unit is further configured to determine the direction and the amount of said rotatable wheel movement.

In some embodiments, said device comprises two of said rotatable wheels that are configured such that the endoscope is adapted to be in contact with both of said rotatable wheels.

Still in other embodiments, said rotatable wheel comprises a channel that is adapted to maintain the endoscope within said channel when the endoscope is placed in said electromechanical tracking device.

Yet in other embodiments, said electrical signal receiving unit is further configured to measure time.

In other embodiments, said electrical signal receiving unit is further configured to record the direction and the amount of movement of said rotatable wheel. In these embodiments, in some instances, said electrical signal receiving unit is further configured to associate a particular video image frame obtained by the endoscope to a particular time or position of the endoscope.

Still yet in another embodiment, said electrical signal receiving unit is configured to identify a blurry video image frame obtained by the endoscope based on a set of quality metrics. For example, based on the speed (i.e., length of endoscope travel divided by time), one can configure the electrical signal receiving unit to identify a blurry image. Such a method is well known to one skilled in the art. See, for example, U.S. Pat. No. 7,894,648, which is incorporated herein by reference in its entirety. This is particular applicable where the electric signal receiving unit comprises a central processing unit or a computer. Such electric signal receiving unit can then be programmed using an algorithm or a flow chart similar to that shown in FIG. 1 to identify and to delete and/or alert the clinician or a doctor who is operating the endoscope of a possible blurry image.

Yet still in another embodiment, said electrical signal receiving unit is operatively connected to a display unit. In this manner, a real-time display can be provided to a clinician or a doctor who is operating the endoscope of a blurry image. Alternatively, the data can be stored and can be processed later to allow a more thorough analysis of the quality of the video and/or the reliability of the endoscope procedure.

Typically, said electrical signal receiving unit is configured to alert a user of a blurry video image frame based on said set of quality metrics. The quality metrics can be any parameter that can influence the video image and/or any parameter that can be used to evaluate the endoscopy procedure as well as the proficiency of the person performing the procedure.

In some embodiments, said electrical signal receiving unit is configured to delete, remove, alert, tag, or not display a blurry video image frame based on said set of quality metrics. Such configuration can be achieved typically by acquiring the images digitally or by converting an analog video to a digital form, for example, by use of a computer. The electrical signal receiving unit can include a software program for automatically analyzing the endoscopy and evaluating the quality of the endoscopy procedure.

Another aspect of the invention provides an apparatus for performing an endoscopy procedure. Such apparatus typically includes (i) an endoscope having a camera for capturing images during an endoscopy procedure; and (ii) an electromechanical tracking device that is described herein and is operatively connected to said endoscope. The electrical signal receiving unit of said electromechanical tracking device generates a set of qualitative metrics data as a function of the endoscopy procedure, said set of quality metrics comprising the direction and the speed of endoscope movement.

The apparatus can further comprise an annular-shaped device comprising a movement detecting element configured to detect torque of said endoscope within said annular-shaped device. In some embodiments, the movement detecting element comprises a spherical trackball (e.g., element 91 in FIG. 9) such that a surface of said spherical trackball contacts the outer surface of said endoscope when said apparatus is in operation. In some instances, the spherical trackball can be configured to detect the pressure exerted by said endoscope. In other instances, the electrical signal receiving unit comprises a central processor unit or is part of a computer. In this manner, the video image of the endoscopy procedure can be digitally captured and or the analog video can be converted to a digital format for quality metrics analysis. The video image can be displayed real-time and/or can be stored for analysis and review at a different time as described herein.

Still in other embodiments, the central processor unit is programmed to determine the torque generated by said endoscope using the data generated by the amount of pressure exerted by said endoscope to said spherical trackball. Such information can be used as additional quality metrics in order to evaluate the quality of endoscopy procedure and/or the proficiency of the person (e.g., clinician or a doctor) who is performing the endoscopy procedure.

Yet in other embodiments, said central processor unit is configured to process the image obtained by said endoscope and identify informative images and non-informative images as a function of said set of qualitative metrics. Based on the set of quality metrics associated with a particular video image frame, the video image frame that does not meet the set of predetermined quality metrics parameters can be deleted, removed, tagged, an alert can be provided for that particular video image. Alternatively, such video image frame(s) can simply be not displayed on a monitor thereby alerting the clinician or the doctor who is performing the endoscopy procedure.

In some instances, said annular-shaped device comprises an elastic portion that is capable of allowing the inner diameter of said annular-shaped device to increase or decrease as a function of the outer diameter of and/or the pressure exerted by (i.e., due to the outer diameter of) said endoscope. In some cases, said annular-shaped device comprises a plurality of said elastic portions. This allows the annular-shaped device to be flexible and adaptable to a variety of endoscope sizes.

Still in other embodiments of the apparatus disclosed herein, said set of quality metrics further comprises change in the speed of endoscope, change in the direction of the endoscope movement, or a combination thereof.

Yet still in other embodiments of the apparatus disclosed herein, said electromechanical tracking device generates a set of qualitative metrics data as a function of elapsed time of the endoscopy procedure.

Still yet another aspect of the invention provides a method for performing an endoscopy procedure on a subject. The method of the invention comprises:
operatively attaching an endoscope to an electromechanical tracking device disclosed herein;
performing an endoscopy procedure on a subject using said endoscope while generating a set of qualitative metrics data as a function of the endoscopy procedure, said set of quality metrics comprising the direction and the speed of endoscope movement during the endoscopy procedure; and
determining the quality of the endoscopy procedure based on said set of qualitative metrics.

In some embodiments, said set of qualitative metrics data is a function of elapsed time of the endoscopy procedure.

With regards to colonoscopy procedure in particular, systems, apparatuses and methods of the invention provide at least three major information: (1) real-time visual feedback indication of image changing velocity and image blurriness; (2) automated summative statistics report provided immediately following the colonoscopy, including withdrawal time, % time of adequate visualization, and a novel graph of dynamics over time; and (3) automated stool coverage analysis for the documentation of bowel preparation. All of these outputs are obtained automatically, thereby allowing colonoscopy quality control in the day-to-day medical practice setting.

### BRIEF DESCRIPTION OF THE DRAWINGS

FIG. 1 is one embodiment of a flowchart for frame blurriness detection by the electromechanical tracking device.

FIG. 2 is a schematic drawing of the front view of one particular embodiment of the endoscope tracking device.

FIG. 3 is a schematic drawing of the back view of the endoscope tracking device of FIG. 2.

FIG. 4 is a schematic drawing of the lateral view of the endoscope tracking device of FIG. 2.

FIG. 5 is a schematic illustration of the opening of the endoscope tracking device of FIG. 2 for the positioning of the colonoscope in the middle section.

FIG. 6 is a schematic illustration of one particular support design for affixing the endoscope tracking device to a surgical bed.

FIG. 7 illustrates the colonoscopy procedure set up including the endoscope tracking device affixed to the bed. The endoscope tracking device is placed in front of the handset, i.e., the torque of the colonoscope.

FIG. 8 illustrates the colonoscopy procedure set up with the endoscope tracking device affixed to the bed using the support 87. The endoscope tracking device is positioned behind the handset.

FIG. 9 shows a schematic illustration of an annular-shaped or a ring-like design of the endoscope tracking device that can be or is intended to be placed at the anus.

FIG. 10 is a cross-sectional view of the annular-shaped endoscope tracking device of FIG. 9.

FIG. 11 is a 3-D illustration of the annular-shaped endoscope tracking device.

### DETAILED DESCRIPTION OF THE INVENTION

The present invention provides a device for tracking an endoscope and a method for using the same. Some aspects of the invention are directed to an apparatus which includes the device of the invention in combination with an endoscope. While the device of the invention can be used in conjunction with any endoscope, such as colonoscope, bronchoscope, gastroscope, laryngoscope, cystourethroscope, esophagogastroduodenoscope, proctosigmoidoscope, etc., for the sake of clarity and brevity, the present invention will now be described in reference to colonoscope. However, it should be appreciated that the device of the invention can be used with all known endoscopes as well as other endoscope that are being or will be developed. Thus, the scope of the invention encompasses use of the device in conjunction with any endoscope.

One of the advantages of the device of the invention is that its placement is simple. The user simply places or attaches the device to a steady surface, so that the colonoscope cord can pass through the middle section of the device. This placement of the colonoscope cord triggers the operation of the embedded position sensors. Another advantage of the device of the invention is that the fabrication process is simple compared to other approaches. In particular, the device of the invention comprises one or more, typically two or more, often two to eight, more often two to six and most often two to four electromechanical tracking sensors. As stated above, use of the device of the invention does not require any modification of conventional colonoscope that are currently available. The electromechanical tracking device is placed externally to the colonoscope. The device of the invention provides and analyzes information about inter alia the movement of colonoscope both forward and backward distance in real time.

Some aspects of the invention provide apparatuses and methods for providing quality metrics of endoscopy procedure, such as colonoscopy. Exemplary quality metrics provided by apparatuses and methods of the invention include, but are not limited to, one or more of the following: quality of image, quantification of bowel preparation and withdrawal velocity that the Colometer system already provides, in addition to the monitoring of the colonoscope direction parameter provided by the electromechanical device disclosed herein.

The Colometer has been used for measuring colonoscopy quality. Briefly, it combines velocity and image blurriness as an output metric. The Colometer offers important advantages with respect to withdrawal time monitoring, which can be part the quality metrics. It is believed that the Colometer can be used as a competitive equivalent with respect to measuring the withdrawal time. However, Colometer provides a relatively simpler, cheaper and faster, reporting quality of colonoscopy relative to withdrawal time monitoring. Furthermore, it is believed that the Colometer is more reliable compared to withdrawal time monitoring, because the Colometer is automated and unalterable. It is also user-friendly, and requires almost no effort from the clinical staff. In addition, its automated method for stool analysis can be more easily correlated with the Ottawa Scale scores, which is the main bowel preparation scale used in Canada, contrasting the Mayo Foundation's system that uses the Boston Bowel Preparation Scale.

With the integration of the endoscope tracking device (i.e., electromechanical tracking system) disclosed herein, the processing time to compute output parameters presented by the Colometer is significantly reduced, since the estimation of velocity and blurriness is very straightforward because the necessary input data can be expressed in terms of distance and time.

Other aspects of the present invention provide a one-piece miniature device that tracks the colonoscope movement. Its placement is simple, since the clinician has to attach the device to a steady surface, so that the colonoscope cord can pass through the middle section of the device triggering the operation of the embedded position sensors. Furthermore, fabrication process for the endoscope tracking device of the present invention is significantly simpler compared to other approaches, since it basically consists of an apparatus with one or several electromechanical tracking sensors. More significantly, the endoscope tracking devices of the present invention can be used with conventional colonoscope as devices of the invention do not require any modifications of the currently available colonoscope because the endoscope tracking device of the invention is placed externally to the colonoscope. In some embodiments, the endoscope tracking device of the invention provides information about the travelled forward and backward distance of endoscope in real time.

Some aspects of the invention comprise an embedded system external to the colonoscope that can automatically provide: a) determination of colonoscope direction; 2) summative statistics following the colonoscopy showing the ratio of forward and backward movements during withdrawal time.

Other aspects of the invention provide methods for evaluating the quality of a colonoscopic procedure. Such methods include passing an endoscopic camera through the lumen of the colon and evaluating information relating to the movement of the colonoscope. From the colonoscope movement, quality metrics associated to the colonoscopy procedure is determined and/or calculated.

Some embodiments of the invention include: a device that can be optionally affixed to the bed and include one or more electromechanical tracking sensors that sense the movement of the colonoscope when the colonoscope cord passes through the middle section of the device; a rotational support affixed to the surgical bed that allows the positioning of the tracking device to facilitate its manipulation by the doctor; and a dashboard (e.g., monitor or a display device) of summary metrics and graphics of the ratio between forward and backward movements after the colonoscopy is completed.

Alternative to said rotational support, a similar approach to track direction and speed (or time) can be implemented in a miniaturized design of an annular-shaped or a ring-like, bearing-based device. Annular-shaped device can be “mounted” at or near the anus of a patient. In general, the electromechanical device provides sensing of the retraction and the advancement of the colonoscope.

The assessment of the colonoscopy would be favorable if the automatically given value is within an already established range. Otherwise, the colonoscopy test would be evaluated as a procedure of poor endoscopy practice, and an advice would be issued to repeat it.

One particular aspect of the invention provides an apparatus for performing colonoscopy. Such an apparatus typically includes: an endoscope having a camera for capturing images during colonoscopy; an endoscope tracking device operatively connected to said endoscope, wherein said endoscope tracking device comprises an electromechanical tracking device adapted to determining the direction and speed of endoscope movement; and an processor operatively connected to said endoscope tracking device for recording direction and speed of endoscope movement and to said endoscope for recording images during colonoscopy. The processor generates data representative of the colonoscopy procedure, and computes a set of quality metrics representative of the quality of the colonoscopy procedure as a function of the colonoscopy procedure data, the set of quality metrics comprising the direction and the speed of endoscope movement.

In some embodiments, the endoscope tracking device further comprises a means for placing said endoscope onto said endoscope tracking device. In one particular embodiment, such means for placing endoscope onto said endoscope tracking device comprises a first wheel; and a second wheel located within proximity of said first wheel such that said endoscope can be snugly fitted between said first wheel and said second wheel.

In some instances, said means for placing endoscope onto said endoscope tracking device further comprises a hinge that is adapted to separate said first wheel from said second wheel during placement of said endoscope between said first and second wheels.

In other embodiments, said endoscope tracking device comprises an annular-shaped device comprising a plurality of movement detecting elements each of which is operatively connected to said electromechanical tracking device such that said endoscope tracking device is capable of further detecting torque of said endoscope within said annular-shaped device.

In some instances, said annular-shaped device comprises at least three movement detecting elements. In some particular cases, said movement detecting elements comprise a trackball or a spherically-shaped element such that the surface of each of said spherically-shaped element contacts the outer surface of said endoscope.

Yet in other embodiments, each of said spherically-shaped element is operatively connected to an electromechanical device to allow determination of the direct and distance of endoscope movement as well as the amount of pressure exerted by said endoscope. In this manner, the processor can be programmed to determine torque generated by endoscope. For example, by using the data generated by the amount of pressure exerted by said endoscope to each of said spherically-shaped element, the processor can determine the amount of torque generated by endoscope as well as the direction of endoscope movement.

Still in other embodiments, said annular-shaped device comprises an elastic portion that is capable of allowing the inner diameter of said annular-shaped device to increase or decrease depending on the outer diameter of said endoscope. Such flexibility allows one to easily insert endoscope into said annular-shaped device. In some instances, said annular-shaped device comprises a plurality of said elastic portions.

In other embodiments, said processor processes the images to identify informative images and non-informative images as a function of said colonoscopy procedure data. Yet in other embodiments, said processor generates a rating of the colonoscopy procedure as a function of the set of quality metrics.

Still in other embodiments, the set of quality metrics further comprises change in the speed of endoscope, change in the direction of the endoscope movement, or a combination thereof.

Devices, systems, apparatuses and methods described herein can be used to provide automated, real-time, feedback-based, and non-operator dependent information about the dynamics and the level of the bowel preparation of a colonoscopy procedure which can be used to evaluate the reliability of the result of colonoscopy procedure and/or the proficiency of the person performing such a procedure.

In some embodiments, the quality metrics includes a colometer system. Such a system can be a software-based, automated image analysis tool to improve the quality of the screening colonoscopy.

The Colometer is a real-time feedback tool for colonoscopic procedures. It offers an indicator for image velocity and blurriness that is displayed on the endoscopist's monitor in real-time. This tool it is also capable of showing a summarized report after each screening test. It is calculated that the number of blurry images contained in a video ranges from 37% to 60% of total video length, depending on different factors including the endoscopist's skills and the level of bowel preparation in every patient. Several different methods have been proposed for the assessment of colonoscopy videos blurriness. However, there is no report of an algorithm for real-time implementation. The Colometer approach for blurriness measurement consists of an instant calculation of the image variance in a frame-by-frame evaluation setup.

However, velocity change monitoring integrated within the Colometer is a practical tool used to evaluate the adequate time spent to correctly examine the colon. Presently, clinicians do not have a feedback of this metrics until the end of the screening test and, therefore, this feedback does not influence the quality of the procedure. The Colometer offers such real-time feedback for the doctor during colonoscopy and a final summary report for patient awareness after the procedure, which can objectively assess its quality.

The Colometer also displays summative statistics in a final report. This report includes the withdrawal time, % of clear visualization, % of overall bowel preparation and an image dynamics-time graph. The objective of this graph is to show the endoscopist when s/he is going too fast during colonoscope withdrawals. Finally, the Colometer can be easily implemented in any colonoscope with a digital video output, since it employs widely available video processing technology.

Some aspects of the invention provide an apparatus for the automatic acquisition of parametric values that can be easily correlated to the quality of a colonoscopic or endoscopic procedure. In particular, during a colonoscopy procedure, when it gets difficult to approach certain areas in the lumen, moving the colonoscope forward and backward allows the clinician to have better visualization of the region and to capture more convenient images for evaluation.

In some embodiments, the apparatus of the invention measures the travelled forward and backward distance of endoscope using an endoscope tracking device, which comprises an electromechanical tracking device. The endoscope tracking device is in direct contact with the endoscope cord which freely moves along a rigid section in the middle of the endoscope tracking device. The forward and backward movement is detected by the electromechanical sensor(s) that is operatively connected to the endoscope tracking device. Such a movement, including the distance travelled by the endoscope is recorded. In some embodiments, the torque exerted by the endoscope within the endoscope tracking device is also recorded. Such information can be processed by a central processor unit to provide colonoscopy quality metrics.

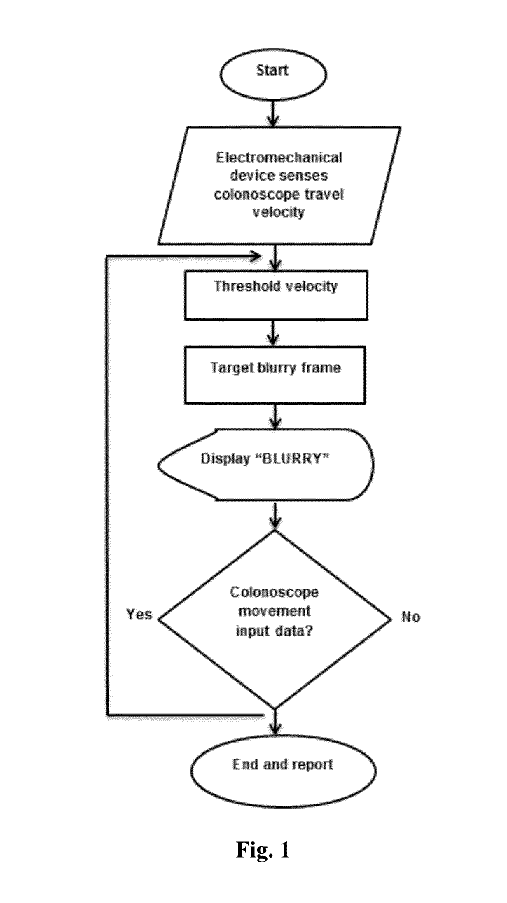

FIG. 1 is a flow chart showing one particular algorithm for frame blurriness recognition using the quality metrics. This algorithm is able to simplify blurriness detection in colonoscopy videos.

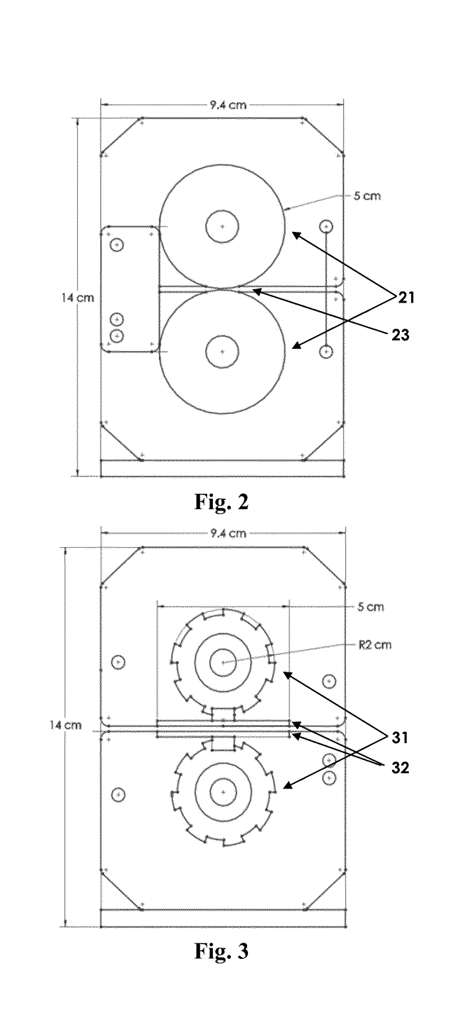

FIG. 2 depicts the electromechanical tracking device that includes two rotating wheels 21′ that are calculatedly positioned leaving a space 23′ in between. This space allows the placement of the colonoscope within the tracking device.

FIG. 3 depicts the back view of the tracking device. The two wheels 31′ get rotated when the colonoscope cord passes through the middle section of the tracking device. This motion is measured by electromechanical sensors 32′ and wirelessly displayed to the user in real time by additional electronic components within a case attached to the device.

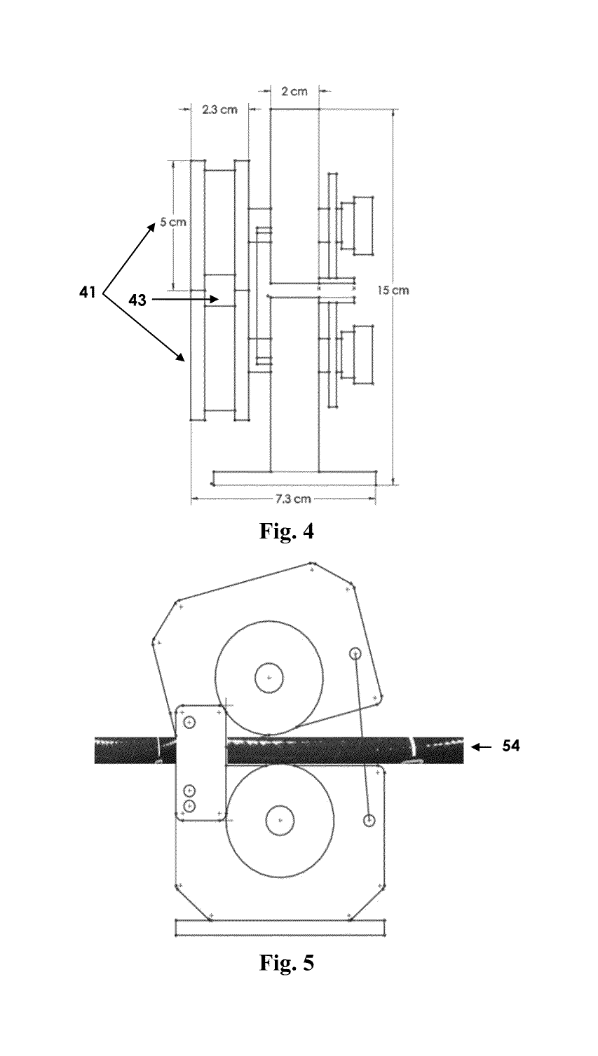

FIG. 4 shows the lateral view of the electromechanical tracking device, where it is perceived the space 43′ in between the two wheels 41′ for the positioning of the colonoscope. Overall dimensions of the device are shown as well.

FIG. 5 depicts the electromechanical tracking device in an opened fashion indicating how the colonoscope 54′ is intended to be placed in the middle section of the device.

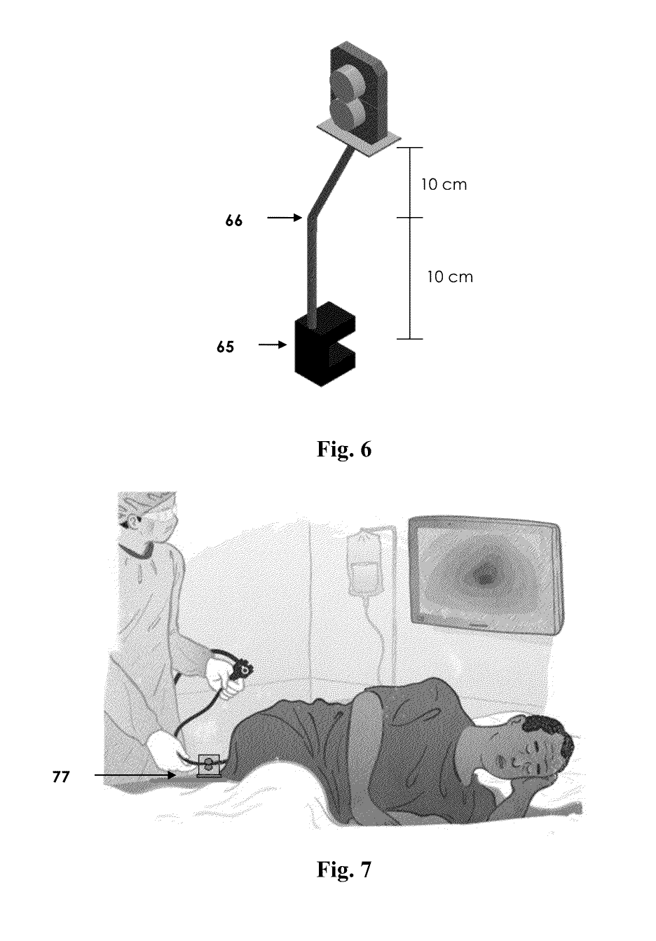

FIG. 6 depicts the support of the electromechanical tracking device that is affixed to a hospital bed using a clamp 65′. The support consists of a metallic arm that relies on the joints 66′ to fully turn around and place the colonoscope at the most convenient site for the doctor. In addition, dimensions of the entire device are shown.

For practicability, different sites for the electromechanical device positioning in the surgical room are proposed. FIG. 7 depicts a colonoscopy procedure where the device is positioned in front of the torque of the colonoscope 77′.

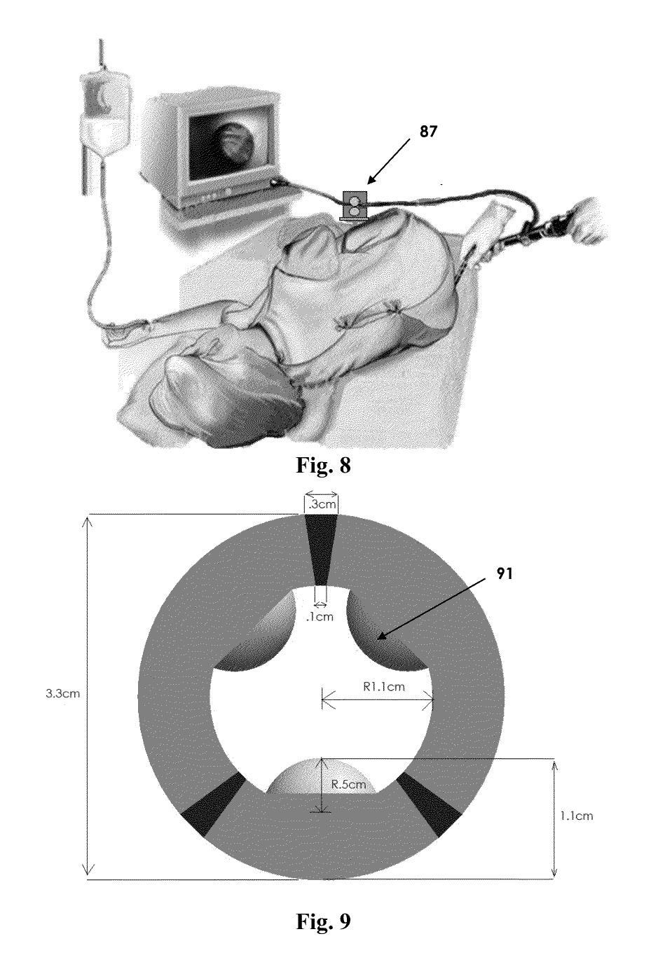

On the other hand, FIG. 8 depicts a colonoscopy procedure and the positioning of the electromechanical tracking device behind the torque of the colonoscope 87′.

A soft and smooth contact between the tracking device and the colonoscope is aimed, so the tracking device doesn't impede the colonoscope maneuvering by the doctor. In FIG. 9, it is shown a miniaturized ring-like (e.g., annular-shaped), bearing-based device that can be held just outside the anus. The device has one or more trackballs or spherical element 91′ that detect the forward-backward motion when the colonoscope passes across the ring and makes the trackball roll. One particular overall dimensions of the ring device are presented in FIG. 9 as well.

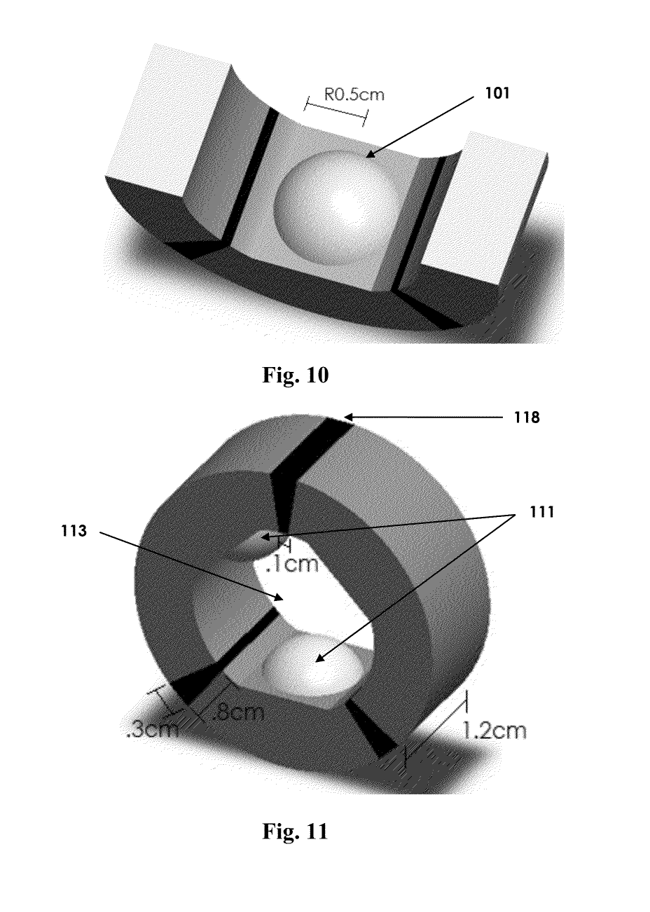

FIG. 10 depicts the transversal section of the ring device mounted at the anus. The device contains one or several protruding balls 101′; each one of them is held by a socket surrounded by the ring. The socket contains the electronic components that record motion when the trackball rolls as a result of the colonoscope movement.

FIG. 11 depicts a 3-D general view of the ring device held at the anus. The ring device slightly embraces the colonoscope, which is positioned in the middle section 113′of the device. The ring diameter can be automatically adjusted in order to assure surface-to-surface contact between the trackball and the colonoscope. This is possible due to some portions of extensible (e.g., elastic) material 118′ in the ring. This material gets stretched when positioning the colonoscope, and shrinks once the colonoscope is placed in. However, the free movement of the colonoscope is still guaranteed since the ball's material 111′ offers no significant friction when being in contact with the colonoscope surface, i.e., it does not significantly hinder the movement of the colonoscope.

Based on the calculation of the ratio between forward and backward movements, the quality of the colonoscopy video can be assessed. For instance, the procedure will be considered of good quality if the given value fits a predetermined standard, otherwise, it will be concluded that forward and backward maneuvers were not conducted in the proper and expected fashion.

In addition, once forward and backward movements of the colonoscope have been recorded throughout the colonoscopy procedure, a further application of the invention described herein is the automatic documenting of the withdrawal time. Furthermore, it is possible to relate a high speed movement of the colonoscope to the formation of non-informative frames, thus allowing their automatic omission from the colonoscopy video. The above-mentioned applications can help to create a colonoscopy video database where the video storage space and, respectively, the necessary time for video assessment, will be significantly reduced.

As discussed above, the set of quality metrics can be used to evaluate and/or to determine the endoscopist's skill and the quality of the procedure. The following are exemplary metrics in relation to a colonoscopy. However, it should be noted that the described metrics are merely examples and the invention is not so limited. Furthermore, the metrics described herein may also be applied to evaluate the quality of other endoscopic procedures in which an endoscopic camera is advanced through a tube or lumen.

One metric is the overall duration of the insertion phase, i.e., insertion time. Another metric is the overall duration of the withdrawal phase, i.e., the withdrawal time. Longer insertion and withdrawal times may indicate that the clinician or the doctor performing the colonoscopy procedure is slowly and carefully advancing the colonoscope through the colon. Still another metric is (i) the clear withdrawal time defined as the duration of the withdrawal phase without out-of-focus frames; and (ii) the ratio of the clear withdrawal time to the overall withdrawal time. Yet another metric is the number of camera motion (e.g., directional) changes and the ratio of the number of camera motion changes to the clear withdrawal time. Still another metric is the fraction of the clear withdrawal time that is spent for close inspections of a colon wall and the ratio of close inspections to global inspections of the colon.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1. An electromechanical tracking device configured for use in an endoscopy procedure, said electromechanical tracking device comprising:
a rotatable wheel configured such that an endoscope is adapted to be in contact with said rotatable wheel, wherein said rotatable wheel is further configured to rotate in proportion to the amount of movement of the endoscope that is in contact with said rotatable wheel;
an electromechanical sensor operatively connected to said rotatable wheel, wherein said electromechanical sensor is configured to generate a first signal when said rotatable wheel moves in one direction and a different signal when said rotatable wheel moves in an opposite direction; and
an electrical signal receiving unit that is configured to receive the electric signal generated by said electromechanical sensor, wherein said electrical signal receiving unit is further configured to determine the direction and the amount of said rotatable wheel movement.

2. The electromechanical tracking device of claim 1, wherein said device comprises two of said rotatable wheels that are configured such that the endoscope is adapted to be in contact with both of said rotatable wheels.

3. The electromechanical tracking device of claim 1, wherein said rotatable wheel comprises a channel that is adapted to maintain the endoscope within said channel when the endoscope is placed in said electromechanical tracking device.

4. The electromechanical tracking device of claim 1, wherein said electrical signal receiving unit is further configured to measure time.

5. The electromechanical tracking device of claim 1, wherein said electrical signal receiving unit is further configured to record the direction and the amount of movement of said rotatable wheel.

6. The electromechanical tracking device of claim 5, wherein said electrical signal receiving unit is further configured to associate a particular video image frame obtained by the endoscope to a particular time or position of the endoscope.

7. The electromechanical tracking device of claim 6, wherein said electrical signal receiving unit is configured to identify a blurry video image frame obtained by the endoscope based on a set of quality metrics.

8. The electromechanical tracking device of claim 7, wherein said electrical signal receiving unit is operatively connected to a display unit.

9. The electromechanical tracking device of claim 8, wherein said electrical signal receiving unit is configured to alert a user of a blurry video image frame based on said set of quality metrics.

10. The electromechanical tracking device of claim 8, wherein said electrical signal receiving unit is configured to delete, remove, alert, tag, or not display a blurry video image frame based on said set of quality metrics.

11. The electromechanical tracking device of claim 1, wherein said electrical signal receiving unit comprises a central processing unit.

12. An apparatus for performing an endoscopy procedure, said apparatus comprising:
an endoscope having a camera for capturing images during an endoscopy procedure; and
an electromechanical tracking device of claim 1 operatively connected to said endoscope,
wherein said electrical signal receiving unit of said electromechanical tracking device generates a set of qualitative metrics data as a function of the endoscopy procedure, said set of quality metrics comprising the direction and the speed of endoscope movement.

13. The apparatus of claim 12 further comprising an annular-shaped device comprising a movement detecting element configured to detect torque of said endoscope within said annular-shaped device.

14. The apparatus of claim 13, wherein said movement detecting element comprises a spherical trackball such that a surface of said spherical trackball contacts the outer surface of said endoscope when said apparatus is in operation.

15. The apparatus of claim 14, wherein said spherical trackball is further configured to detect the pressure exerted by said endoscope.

16-18. (canceled)

19. The apparatus of claim 15, wherein said annular-shaped device comprises an elastic portion that is capable of allowing the inner diameter of said annular-shaped device to increase or decrease as a function of the outer diameter of said endoscope.

20. (canceled)

21. The apparatus of claim 12, wherein said set of quality metrics further comprises change in the speed of endoscope, change in the direction of the endoscope movement, or a combination thereof.

22. The apparatus of claim 12, wherein said electromechanical tracking device generates a set of qualitative metrics data as a function of elapsed time of the endoscopy procedure.

23. A method for performing an endoscopy procedure on a subject, said method comprising:
operatively attaching an endoscope to an electromechanical tracking device of claim 1;
performing an endoscopy procedure on a subject using said endoscope while generating a set of qualitative metrics data as a function of the endoscopy procedure, said set of quality metrics comprising the direction and the speed of endoscope movement during the endoscopy procedure; and
determining the quality of the endoscopy procedure based on said set of qualitative metrics.

24. The method of claim 23, wherein said set of qualitative metrics data is a function of elapsed time of the endoscopy procedure.
